(19) United States
(12) Patent Application Publication
(10) Pub. No.: US 2012/0318261 A1
(43) Pub. Date: Dec. 20, 2012

(54) Valved Holding Chamber With Whistle for the Administration of Inhalable Drugs

(75) Inventors: Michael Newhouse, Hamilton (CA); K. Mosaddeq Hossain, Hillsbrough, NJ (US); Paresh Vasandani, Somerset, NJ (US); Osnan L. Barros-Neto, Hancock, MI (US); Rohinton D. Toddywala, Princeton, NJ (US); Vijay Shukla, Highland Park, NJ (US)

(73) Assignee: NOSTRUM TECHNOLOGY LLC, New Brunswick, NJ (US)

(21) Appl. No.: 13/494,706

(22) Filed: Jun. 12, 2012

Related U.S. Application Data
(60) Provisional application No. 61/498,483, filed on Jun. 17, 2011.

(51) Int. Cl. A61M 15/00; A61M 16/06; A61M 11/04

(52) U.S. Cl. 128/200.23

(57) ABSTRACT

This invention discloses a chamber adapted for the administration of medication from a source of aerosolized drug, such as a metered dose inhaler (MDI) or nebulizer, comprising a conduit with an oval cross section, a mounting for the MDI at the rear, an annular one-way valve at the front in fluid communication with the inhalation airway of the patient that permits air to flow out of the chamber during an inhalation by the patient, but prevents exhaled air from entering the chamber, an exhaust valve in the front section that provides an effective seal during inhalation, and a solid state whistle, wherein the whistle is adapted to making a sound if the air pressure within the chamber is below a predefined threshold. The inventive chamber may be used to directly administer inhaled medication to the mouth of a patient, or it may be used with an inhalation mask.

### CROSS REFERENCE TO RELATED APPLICATION

This application claims priority to U.S. provisional patent application No. 61/498,483, filed Jun. 17, 2011, the entire contents of which are incorporated herein by reference.

### FIELD OF THE INVENTION

This invention pertains to an apparatus for use in the administration of inhaled drugs.

### BACKGROUND

Aerosolized drugs are important medicaments for the treatment of asthma, chronic obstructive pulmonary disease (COPD), other respiratory diseases, and even other non-respiratory conditions, where delivery of a drug substance to the lungs is desired. Drugs delivered directly to the lungs may act locally in the lungs, or be absorbed in the lungs for delivery elsewhere in the body. By the term “aerosolized drugs” is meant a gaseous suspension of fine solid or liquid drug substance that is intended for delivery by inhalation to the lungs of a patient in need of such drug.

A frequently used and inexpensive source of aerosolized drugs are metered dose inhalers (MDI's). They are extremely popular because of their ease of use, and because they can efficiently deliver aerosolized medication directly to the lungs, which is highly advantageous in respiratory conditions. MDI's consist of a pressurized canister containing a liquid or powdered drug product and a propellant, and include an actuation device, typically a Meshberg valve, and a valve stem as outlet. There is also typically an adapter with a mouthpiece. The valve stem is seated in a receptacle in the adapter. The valve stem and valve dispense a dose of the drug when the canister is depressed within the adapter. In a simple embodiment, the patient uses the mouthpiece of the adapter directly to inhale medication. A feature of these devices is that the patient must coordinate an inhalation with actuating the MDI. This coordination is a problem for many patients. Additionally, mouth or throat irritation, hoarseness and fungal infection can be a problem due to deposition of a large proportion of the drug or propellant particles in the mouth or throat, rather than the lungs.

Another commonly used source of aerosolized drugs are nebulizers, which may, in an embodiment, have a reservoir containing a drug suspended or dissolved in an aqueous solution. The solution is nebulized by capillary action in a jet of air or oxygen that generates a mist or vapor of suspended atomized droplets, which is conveyed to the mouth of the patient through a tube and a mouthpiece or a mask. Other aerosol generators make use of ultrasonically vibrating piezo-electric crystals, a vibrating mesh or relatively high pressure to force the drug solution or suspension through very small holes.

Valved aerosol reservoirs also known as valved holding chambers or spacers coupled to MDI's are well known in the art as having certain advantages. In some patient populations, their use is mandatory. In one aspect, a simple chamber coupled to an MDI acts as a spacer or holding chamber to improve the mixing of drug from an MDI with air. Also, with a simple spacer, larger particles drop out of the effluent prior to inspiration by the patient. This results in less deposition of drug in the mouth and throat, which is undesirable, and improved delivery of the aerosolized drug to the lungs. A simple spacer device is disclosed, for example, in WO 2004/091704.

In another aspect, a chamber may have a one-way inhalation valve, as disclosed for example in U.S. Pat. Nos. 5,012,804; 5,042,467; and 6,026,807. By the use of an inhalation valve, the user does not need to coordinate their inhalation with the source of aerosolized drug, such as an actuation of an MDI. This is important, for example, for inexperienced users, incompetent users, or children. The majority of the effluent (80% approximately) from the MDI can remain suspended within the chamber for up to 20 seconds before inhalation, and deliver an effective dose to the lungs Even if the patient exhales prior to inhaling.

In another aspect, a chamber with a one-way inhalation valve can be used with an inhalation mask. With an inhalation mask, the patient does not need to put their lips around a mouthpiece. This is particularly useful with small children or incompetent patients. Such masks have been disclosed, for example, in U.S. Pat. No. 5,645,049.

In another aspect, chambers have been equipped with a high-flow alarm in the form of a reed that makes a sound if the patient is inhaling too rapidly. Such high-flow alarms are disclosed, for example, in U.S. Pat. Nos. 5,042,467, 5,848,588, and 6,523,536. Low and moderate inhalation velocity provides superior drug delivery to the small airways of the lungs. High inspiratory flow velocity causes impaction of the larger particles in the mouth, throat and first few bronchial divisions, whereas more moderate inspiratory velocity allows even larger particles to bypass the upper respiratory tract and “float” into the small airways and alveoli. This is because of the decreased turbulence in the larger central airways of the lungs. The trachea has a cross-sectional area (in adults) of approximately 2.5 cm², but the surface area of the aveoli is estimated to be 80 m², so there is a huge amount of volumetric expansion in the lungs. Thus, there is no need for rapid flow rates to achieve an even distribution of drug throughout the lungs. Indeed, at a very low flow of about 0.1 L/sec particles as large as 6 microns can quite efficiently reach the small airways and alveoli.

The inspiratory flow velocity can therefore be an important consideration depending on the particle size of the product. Some MDI's have particles in the range of 0.5 to 3 μm, and at such small particle sizes, the flow rate is less critical. Other MDI's have larger particle sizes for which the flow rate is more critical. In the case of drugs intended for pulmonary conditions (e.g., asthma or COPD), a relatively even distribution of drug particles throughout the lung, including large and small airways and alveoli, is highly desirable. By contrast, in the administration of inhaled drugs for non-pulmonary conditions, for example, inhaled insulin, delivery of the drug to the alveoli may be more important than to the airways, since presumably more drug absorption occurs in the enormous surface area of the terminal airways and alveoli than from the conducting airways of the lung.

### SUMMARY

This invention discloses an audible biofeedback system for the administration of inhalable drugs that includes a chamber adapted for the administration of medication from an MDI, comprising a generally cylindrical chamber with a circular or oval cross section, a mounting for the MDI at the rear, a one-way valve at the front in fluid communication with the inhalation airway of the patient that permits air to flow out of the chamber during an inhalation by the patient, but prevents exhaled air from entering the chamber, an exhaust valve in the front section that provides an effective seal during inhalation, and a solid state whistle, wherein the whistle is adapted to making an audible tone (i.e., a sound) if the air flow through the whistle exceeds a predefined threshold.

The inventive chamber may be used to directly administer inhaled medication to the mouth of a patient, or it may be used with an inhalation mask.

### DESCRIPTION OF THE DRAWINGS

FIG. 1A is an exploded view of an embodiment of the inventive chamber, valves, and whistle.

FIG. 1B is an exploded view of an alternative embodiment of the inventive chamber, valves, and whistle.

FIG. 2A is a side elevation view of an embodiment of the inventive chamber, valves, and whistle.

FIG. 2B is a top elevation view of an embodiment of the inventive chamber, valves, and whistle.

FIG. 2C is an elevation of the front or anterior end of an embodiment of the inventive chamber, valves, and whistle.

FIG. 2D is an elevation of the back or posterior end of an embodiment of the inventive chamber, valves, and whistle.

FIG. 2E is a perspective view (from the rear) of an embodiment of the inventive chamber, valve, and whistle.

FIG. 2F is a perspective view (from the front) of an embodiment of the inventive chamber, valves, and whistle.

FIG. 3 is a cross section view of an embodiment of the inventive device lengthwise, along a horizontal axis with the upper portion cut away.

FIG. 4 is a cross section view of an embodiment of the inventive device lengthwise along a vertical axis

FIG. 5A is an exploded view of an embodiment of the solid state whistle sub-assembly.

FIG. 5B is an exploded view of an alternative embodiment of the solid state whistle sub-assembly.

FIG. 6 shows air flows in a cross section of an embodiment of the inventive device during the inhalation cycle.

FIG. 7 shows air flows in a cross section of an embodiment of the inventive device during the exhalation cycle.

FIG. 8 shows the inventive chamber coupled to a breathing mask.

### DETAILED DESCRIPTION

This invention provides an audible biofeedback system that includes chamber for use with inhalable drugs, in particular for drugs administered from a source of aerosolized drugs, such as a metered-dose inhaler (MDI) or nebulizer. The inventive chamber provides the benefits of mixing suspended or aerosolized drug substance with air in a manner known to provide improved drug delivery to the lungs, and minimizing deposition of drug substance in the mouth and throat of the patient using the device. Such deposition of drug in the mouth and throat is undesirable, and may cause local irritation, hoarseness and other side effects, for example fungal infection (thrush) in the mouth or throat of users of inhaled corticosteroids. The inventive chamber also permits the use of MDI's with inexperienced users, children, or incompetent patients, who cannot coordinate their breathing with the actuation of an MDI, or who cannot follow instructions on the use of an MDI. Furthermore, the inventive chamber permits MDI inhalers to be used with inhalation masks, which is required for some patients.

In addition to MDI's, the inventive chamber can be used with other sources of aerosolized drugs, to act as a reservoir for nebulizer-generated (non-metered) aerosol to store the generated aerosol during the patient's expiration for instance. This would be useful to minimize waste of medication and contamination of the room air that could cause sensitization of caregivers with some drugs.

As defined herein, the anterior section of the disclosed audible biofeedback system apparatus is the section closest (proximate) to the mouth or face of the patient, and the posterior section is the part of the apparatus that is distal to the patient, or at the rear of the apparatus.

The apparatus of the audible biofeedback system has a posterior section comprising a mounting 30 for an MDI device. The anterior section of the apparatus may include a conical front cover 20 with an outlet airway 22 for conducting the aerosolized drug to the mouth or nose (if a breathing mask is used) of a patient. The airway conducts gases including air and suspended, aerosolized, or vaporized medication into the mouth of a patient during inhalation. In an embodiment, the drug airway may be in fluid communication with an inhalation mask that conducts air and suspended, aerosolized, or vaporized medication into the nose or mouth of a patient.

The anterior section of the chamber has a one-way inhalation valve that permits air and suspended, aerosolized, or vaporized medication to flow out of the interior of the chamber into the nose or mouth of a patient via the chamber outlet, but prevents exhaled air from flowing back into the chamber. The anterior portion of the inventive chamber also includes a one-way exhalation valve, which in an embodiment is a flap with a seat in a housing affixed to the chamber. During inhalation the one-way exhalation valve prevents exterior air flow from entering the chamber. During exhalation, the inhalation valve prevents exhaled air from entering the chamber, but the exhalation valve opens to vent the exhaled air outside of the chamber device.

In an embodiment, the inventive chamber has a solid state whistle that may be situated in the posterior section that provides an indication of an appropriate inspiratory effort or appropriate inspiratory flow velocity (flow rate during inhalation). The whistle is designed to make a sound only if the inspiratory flow velocity through the chamber is exceeds a predetermined flow rate (for example, 30 L/min or less). That is, the whistle only produces an audible tone if the pressure within the chamber falls below a predetermined level, which would occur if the patient inhales too vigorously. Thus, the whistle is an audible biofeedback device, informing the user to change their inhalation effort to obtain an optimal inhalation velocity. If the patient inhales too vigorously, the delivery of drug particles (in the aerosol) to the small airways is not as efficient as at low inspiratory velocities.

Prior art flow-alarm whistles, such as disclosed in U.S. Pat. Nos. 5,042,467 and 5,848,588, employ a reed and are distinguished from the inventive whistle here. The inventive whistle has no reed or moving parts. The inventive whistle comprises a hollow cylinder with orifices at each end that operates on the Helmholtz principle. The airflow at which the whistle makes a sound can be tuned to make a sound based on different flow rates. The tuning of the whistle is based on changes to the shape of the whistle and the size of the orifices. Thus, changing the interior diameter of the cylinder, the length of the cylinder, and the size of each orifice will change the flow rate through the whistle at which an audible tone is produced. The optimal flow rate at which the whistle makes a tone to alarm the user that his or her inspiratory effort is too strong depends on the drug being employed and the particular MDI. Thus, different drug products and MDI products will require different whistles with different flow rates required to produce a sound. In various embodiments, the whistle may be optimized to make a sound at an airflow of about 20 L/min, 28 L/min, or 52 L/min.

In an embodiment, the one-way inhalation valve is a duckbill valve.

In an embodiment, the one-way inhalation valve is an annular valve that provides improved air flow, less dead space, and operates effectively at very low inspiratory flow velocities, making it suitable for use with children. Such an annular valve may have a lockdome secured to a post connected to the conduit body, a rubber disk diaphragm, and a valve seat. The valve seat supports the diaphragm and provides a seal during exhalation. The post is centered in a flow conditioning dome. The flow conditioning dome is a solid or hollow dome facing the rear that provides a streamlined airflow. Two brackets support the dome on the conduit body. U.S. Pat. No. 7,201,165 discloses a similar dome but uses a duck bill valve. The inventive valve has the flexible annular disk that opens concentrically toward the user during inhalation, and is forced back into the seat during exhalation.

In an embodiment, this invention provides an audible biofeedback system for the administration of an inhalable drug through an airway to a patient in need of such inhalable drug, comprising an apparatus adapted for the administration of inhalable drug having an anterior section and posterior section, wherein said apparatus comprises a chamber interposed between the anterior and posterior section, where said chamber comprises a generally cylindrical member; a mounting for a metered dose inhaler in the posterior section of the apparatus; a generally cylindrical inhalation airway suitable for insertion into the mouth of a patient in the anterior section of the apparatus; a one-way inhalation valve at the anterior end of the conduit in fluid communication with the inhalation airway that permits gases containing the suspended drug droplets or particles to flow out of the chamber during an inhalation by the patient, but prevents exhaled air from entering the chamber; an exhaust valve in the anterior section that provides an effective seal during inhalation but allows exhaled air to vent away from the face of the patient; and a solid state whistle, wherein the whistle is in fluid communication with the chamber, and is adapted to making a sound if the air flow through the whistle exceeds a predefined threshold.

In an embodiment, the conduit comprises a generally circular cross section. In an alternative embodiment, the conduit comprises a slightly elongated dimension along a horizontal cross-sectional axis to provide a generally oval cross section.

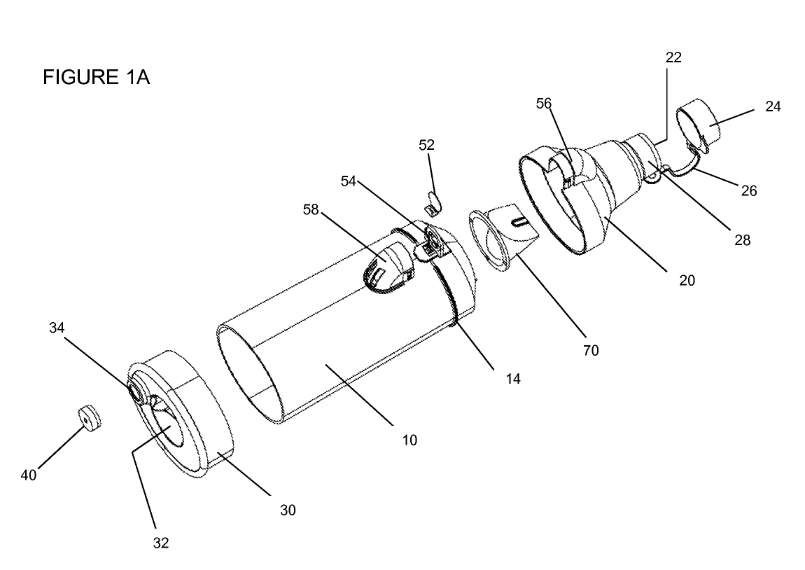

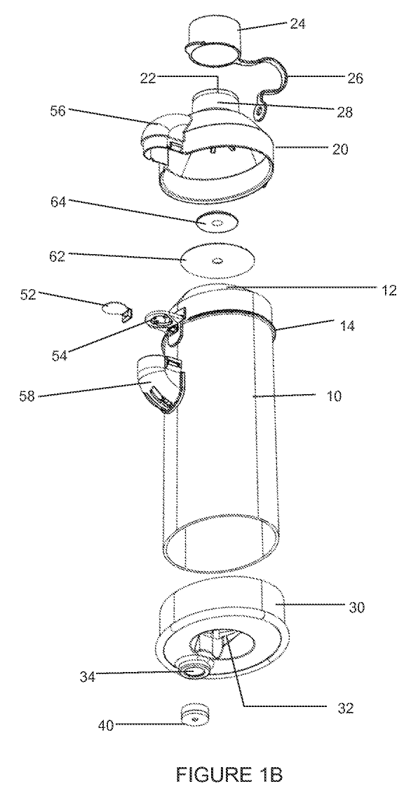

Various embodiments of the audible biofeedback system are shown in the figures. FIGS. 1A and 1B show exploded views of alternative embodiments of the entire apparatus. The conduit 10 spans the posterior MDI mount 30 and the anterior conical cover 20. In an embodiment, as illustrated in the figures, the conduit cross section is shown with an oval shape, with the top and bottom pushed in relative to the horizontal axis. This shape is similar to modern feeding bottle designs for infants. It is believed that infants and small children can grasp the conduit more easily with an oval cross section, as opposed to a perfectly round cross section. Also, the oval shape is believed to aid in removing more of the larger particles that would sediment over a shorter distance due to gravity.

The cover 20 is held in place with the aid of collar 14, which may be an integral part of the conduit member. The cover may also have protective cap 24, secured to the chamber with tether 26, which may be a flexible plastic strip. The interior portion of 20 is airway 22, which is the pathway for the drug substance to the mouth or nose (in the case where an inhalation mask is used) of the patient.

The cover in the illustrated embodiment includes an external exhalation valve assembly 50 including conduits 56, 58, valve diaphragm 52, and valve seat 54. In the embodiment illustrated in the figures, conduit 56 includes a nested coupling with conduit 58, with diaphragm 52 and seat 54 within the airway created by 56 and 58 approximate location of the coupling. Conduit 56 is in fluid communication with airway 22, so that exhaled air which is blocked by the one-way inhalation valve is shunted into conduit 56.

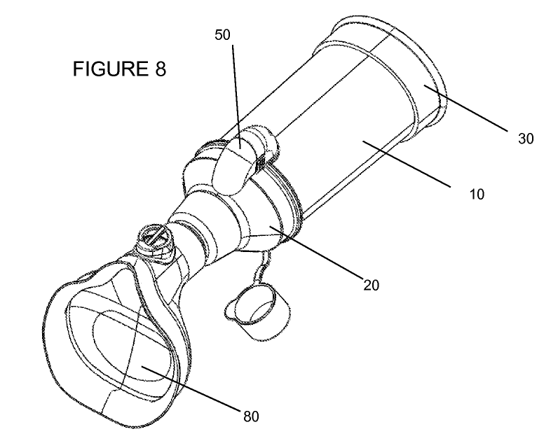

In an embodiment shown in the Figures, the drug outlet 28 has a circular cross section, and can function directly as a mouthpiece, being inserted directly into the mouth of the user. In an embodiment, a disposable mouthpiece (not shown) may be fitted over the drug outlet 28, for example fabricated from cardboard or inexpensive plastic, to prevent a patients lips from directly touching the mouthpiece. This disposable member is particular desirable if the inventive apparatus is used in a clinic, because it allows audible biofeedback system for the administration of an inhalable drug to remain fairly clean between patients. In an embodiment (FIG. 8), an inhalation mask may be fitted over outlet 28.

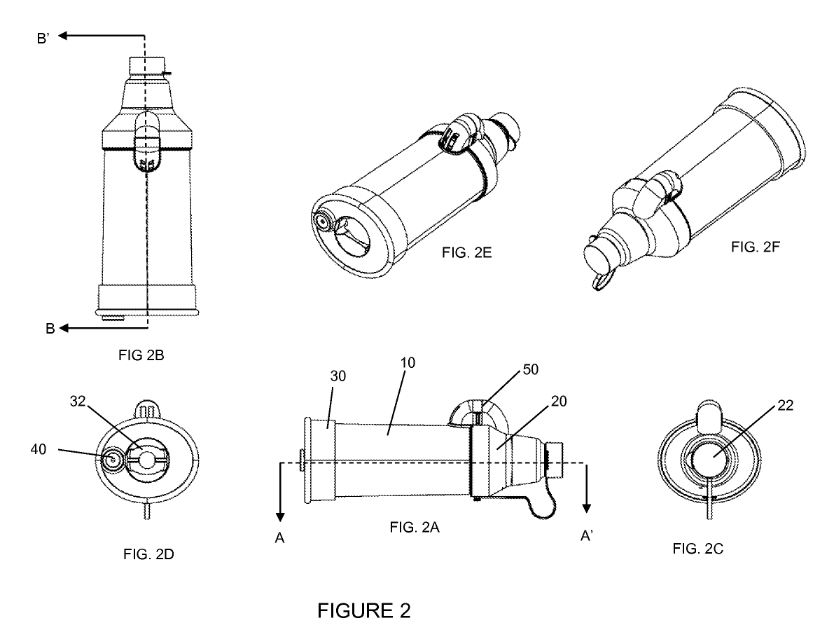

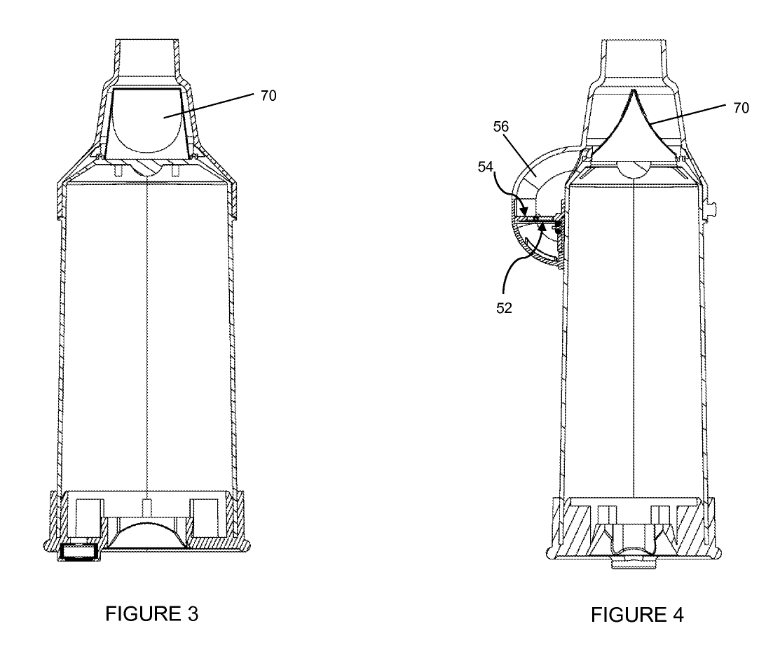

FIGS. 2A-2F show various exterior views of an embodiment of the complete apparatus described herein with an oval-cross-section conduit and duck bill inhalation valve. FIGS. 3 and 4 show cross sectional views of the complete chamber with a duck bill inhalation valve. FIG. 3 is a view of the bottom of the complete apparatus along line A-A′ (FIG. 2A), as if the top of the apparatus as depicted in FIG. 2A was cut away. FIG. 4 is a cross section along the axis defined by line B-B′ (FIG. 2B), as if the right side of the apparatus in the view of FIG. 2B was cut away. The oval cross section profile of conduit 10 can be seen from FIGS. 3 and 4 taken together. Thus, conduit 10 is clearly wider in the axis parallel to the page in FIG. 3 than in FIG. 4.

The exhalation valve comprises diaphragm 52 mounted on the posterior face of seat 54. Diaphragm 52 has a positional bias towards the seat 54. This bias is depicted in cross-sectional view of FIG. 4, showing that in a resting state, diaphragm 52 is generally seated in contact with seat 54. During inhalation, diaphragm 52 is pulled toward the seat by the air flow, and prevents fresh air from entering airway 22 from the exhalation valve 50. During exhalation, the diaphragm is pushed toward the rear allowing exhaled air to vent in a direction away from the patient's face via slits illustrated in conduit portion 58. FIG. 7 shows the air flow through the exhalation valve, including the fluid communication of the exhaled air with conduit 56, and the diaphragm 52 flexing in the posterior direction to allow the exhaled air to escape out of the apparatus through vents in conduit 58.

In an embodiment, the inhalation valve is annular, and includes neck 12 with brackets 68 that support post 61 and flow conditioning dome 66. The inhalation valve diaphragm 62 is secured with lockdome 64. Lockdome 64 in turn is secured to post 61. During inhalation, diaphragm 62 flexes anteriorly due to the air flow, allowing air in the chamber with aerosolized drug to flow into airway 22. The air containing suspended drug flows around the flow conditioning dome 66 and into the airway 22. During exhalation the exhaled air forces diaphragm 62 against its seat, sealing the valve and preventing exhaled air from entering the chamber 11. The exhaled air flows into conduit 56 and is vented via exhaust valve 50.

In an embodiment, the inhalation valve is a duck bill valve 70, seated on a collar that may be integral with conduit member 10, and held in position with suitable bracket that may be integral with cap member 20. In some instances, and with some drug products, the duck bill valve embodiment has been found to give better air flow out of conduit 10 into airway 22. During exhalation, as shown in FIG. 7, the duck bill valve makes a secure seal preventing expired air from entering conduit 10. The sealed duck bill valve forces the expired air out of valve 50 via posteriorly flexing diaphragm 52.

In an embodiment, the posterior portion of the inventive apparatus has mounting 30 that caps the posterior opening of conduit 10. Mounting 30 is designed and shaped so that an MDI fits into opening 32. Opening 32 firmly supports an MDI thus directing the aerosol towards the inhalation valve and may include additional vents for air to enter the chamber during inhalation. Whistle 40 may also be mounted in opening 34, an orifice integral with mounting 30.

The audible biofeedback system described herein may include whistle 40 with no moving parts, in contrast to prior art whistles in MDI chambers that contain a reed. In an embodiment, the whistle 40 is a cylinder situated in the posterior of the audible biofeedback system apparatus, with the longitudinal axis of the whistle cylinder parallel to the longitudinal axis of the conduit 10, with a disk on each end of said cylinder, with an orifice in each disk.

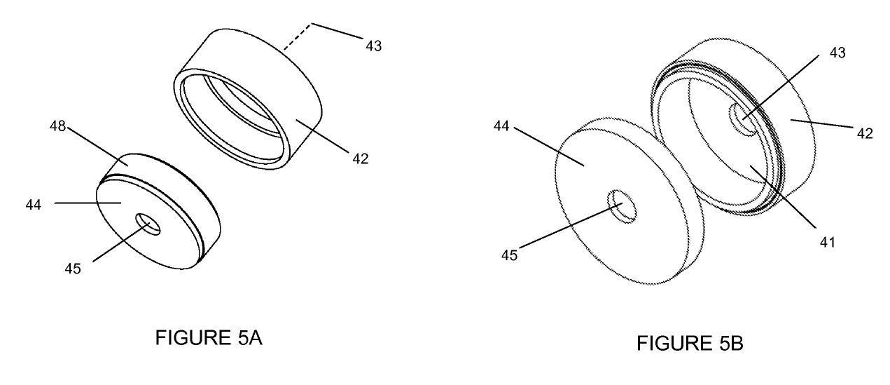

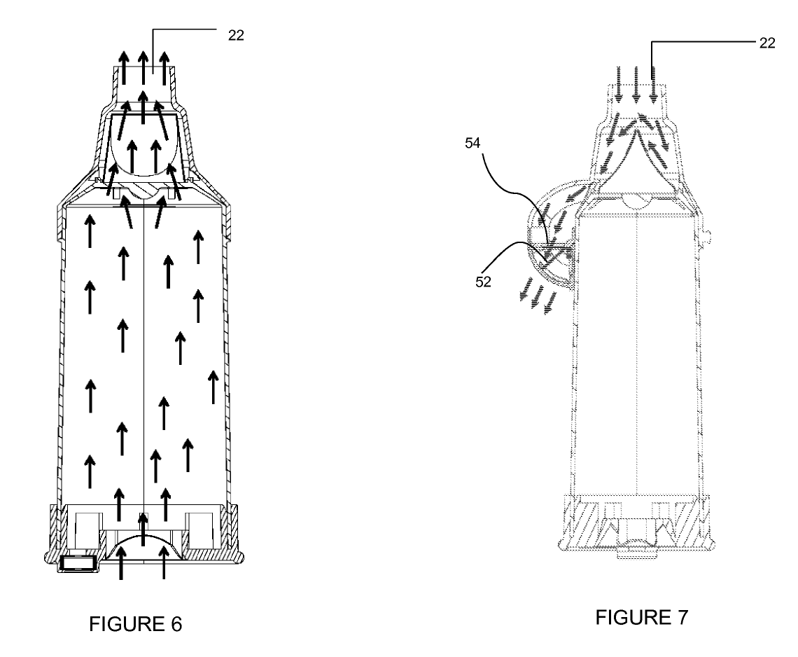

Expanded views of whistle 40 are shown in FIGS. 5A and 5B. The embodiment in FIG. 5A has a cap member including face 44 and cylindrical wall 48 that nests in base member 42. Face 44 has an orifice 45 therein. The base member includes face 41 with orifice 43 therein. The embodiment of FIG. 5B is a slightly different configuration in which the cap member including face 44 fits over and around a lip on base member cylindrical wall 42. In either of the configurations illustrated, which are not intended to be limiting, the assembled whistle comprises a hollow cylinder with flat face sections and each end of the cylinder, with an orifice (43 and 45) in each flat face.

By adjusting the dimensions of each of these parts, that is, the diameter of the 42/44, the distance between 42 and 44, and the size and shape of the orifices 43 and 45, the characteristics of the whistle can be varied. Specifically, the air flow (in mL/sec) at which the whistle makes a tone can be modulated, and the frequency of the tone can be modulated.

In an embodiment, the inventive chamber may be used for the administration of inhaled drugs by a patient. The patient may place their lips directly on exhaust tube 28 and use the chamber to directly inhale the suspended drug. In an embodiment, a disposable plastic or cardboard sleeve may be employed so the patient's lips do not directly touch the audible biofeedback apparatus. In another embodiment, the patient may use an inhalation mask coupled to tube 28 to inhale the medication, as illustrated (for example) in FIG. 8. Several such inhalation masks are known in the art. A preferred mask is disclosed in co-pending application Ser. No. ______, which provides an airway aligned with the nose of the patient, and a contoured mouth section to minimize dead space within the mask.

1. An audible biofeedback system for the administration of an inhalable drug through an airway to a patient in need of such inhalable drug, comprising
a. an apparatus adapted for the administration of inhalable drug having an anterior section and posterior section, wherein said apparatus comprises a chamber interposed between the anterior and posterior section, where said chamber comprises a generally cylindrical member;
b. a mounting for a metered dose inhaler in the posterior section of the apparatus;
c. a generally cylindrical inhalation airway suitable for insertion into the mouth of a patient in the anterior section of the apparatus;
d. a one-way inhalation valve at the anterior end of the conduit in fluid communication with the inhalation airway that permits gases to flow out of the chamber during an inhalation by the patient, but prevents exhaled air from entering the chamber;
e. an exhaust valve in the anterior section that provides an effective seal during inhalation but allows exhaled air to vent away from the face of the patient; and
f. a solid state whistle, wherein the whistle is in fluid communication with the chamber, and the whistle is adapted to make an audible tone if the air flow through the whistle exceeds a predefined threshold.

2. The audible biofeedback system of claim 1 wherein the conduit comprises a generally circular cross section.

3. The audible biofeedback system of claim 1 wherein the conduit comprises a slightly elongated dimension along a horizontal cross-sectional axis to provide a generally oval cross section.

4. The audible biofeedback system of claim 1 wherein the whistle has no moving parts.

5. The whistle of claim 4, wherein the whistle comprises a cylinder with a longitudinal axis parallel to the longitudinal axis of the chamber, with a disk on each end of said cylinder, with a hole in each disk.

6. The audible biofeedback system of claim 1 wherein the predefined threshold at which the whistle makes an audible tone is greater than about 20 L/min.

7. The audible biofeedback system of claim 1 wherein the predefined threshold at which the whistle makes an audible tone is greater than about 28 L/min.

8. The audible biofeedback system of claim 1 wherein the predefined threshold at which the whistle makes an audible tone is greater than about 52 L/min.

9. The audible biofeedback system of claim 1 wherein the whistle is situated in the posterior section of the apparatus.

10. The audible biofeedback system of claim 1 wherein the one-way inhalation valve comprises a flexible plastic disk diaphragm mounted on a valve seat, and with a disk lock dome axially affixed thereto to secure the diaphragm to the valve seat.

11. The valve of claim 10, wherein the lock dome has a geometrically domed profile in the front-facing direction.

12. The valve of claim 10, wherein the valve seat has a geometrically domed profile in the rear-facing direction.

13. The audible biofeedback system of claim 1 wherein the one-way inhalation valve comprises a duck bill valve.

14. The audible biofeedback system of claim 1 wherein the metered dose inhaler mounting comprises flexible plastic and is shaped to permit air flow into the chamber around the MDI device.

15. The audible biofeedback system of claim 1 wherein the airway is inserted directly into the mouth of the patient during administration of medication.

16. The audible biofeedback system of claim 1 wherein a breathing mask is adapted to fit on to the airway.

17. A method of administering inhalable medication to the lungs of a patient, wherein the medication is provided by a source of aerosolized drug, using the apparatus of claim 1.

18. The method of administering inhalable medication to the lungs of a patient of claim 17, wherein the source of medication is a metered dose inhaler; wherein said metered dose inhaler is actuated to provide aerosolized or suspended medication in the chamber; and wherein the patient inhales said aerosolized or suspended medication without the production of an audible tone from the whistle.

19. The method of administering inhalable medication to the lungs of a patient of claim 17 wherein the airway of the audible biofeedback system is inserted directly into the mouth of the patient in need of inhalable drug and.

20. The method of administering inhalable medication to the lungs of a patient of claim 17 wherein a breathing mask is adapted to fit on to the airway of the audible biofeedback system and the medication is administered through the breathing mask to the mouth or nose of the patient in need of inhalable drug.
